(19) United States
(12) Patent Application Publication
(10) Pub. No.: US 2024/0197604 A1
(43) Pub. Date: Jun. 20, 2024

(54) COMPOUNDS WITH COOLING SENSATION PROPERTIES

(71) Applicant: Givaudan SA, VERNIER (CH)

(72) Inventors: Corinne BAUMGARTNER, Hinwil (CH); Nicolas COCITO ARMANINO, Baden (CH); Thierry GRANIER, Duebendorf (CH)

(21) Appl. No.: 18/286,525

(22) PCT Filed: Apr. 21, 2022
(86) PCT No.: PCT/EP2022/060485

§ 371 Date: Oct. 11, 2023

(30) Foreign Application Priority Data
Apr. 21, 2021 (GB) 2105661.9

(51) Int. Cl. A61K 8/49; A61K 31/454; A61Q 11/00; C07D 401/04

(52) U.S. Cl. CPC A61K 8/4946; A61K 31/454; A61Q 11/00; C07D 401/04; A61K 2800/244

(57) ABSTRACT

Disclosed are TRPM8 modulators for achieving a cooling effect on skin and mucousa.

### TECHNICAL FIELD

The present invention relates to a particular class of compounds capable to activate TRPM8 ion channels. It further relates to the use of said compounds for inducing a sensation of coldness, and to consumer products comprising these compounds.

### BACKGROUND

TRPM8 (transient receptor potential melastatin member 8, also known as Trp-p8 or MCR1) is activated by innocuous cool and thus plays an important role as sensor for temperature. The channels are widely distributed in different tissues (such as human skin and mucosa (such as oral mucosa, throat mucosa, and nasal mucosa), male urogenital tract, lung epithelium cells and artery myoctes). They are Ca²⁺-permeable, nonselective cation channels that exhibit polymodal gating mechanisms, being activated by innocuous cool to cold temperature, membrane depolarization, and molecules which are known as cooling agents including natural and synthetic compounds. The receptor was described for the first time in 2002 as cold receptor in a number of publications.

The present invention is based on the finding that a particular class of compounds can be used to drive a cooling response when brought into contact with TRPM8 receptor in-vitro and in-vivo.

Compounds providing a cooling sensation have for a long time played an important role in the flavor and fragrance industry in order to produce an association with freshness and cleanliness. Cooling compounds are widely used in a variety of products such as foodstuffs, tobacco products, beverages, dentifrices, mouthwashes, toothpastes, and toiletries. The cooling sensation provided contributed to the appeal and acceptability of consumer products. In particular, oral care products, such as dentifrices and mouthwashes are formulated with coolants because they provide breath freshening effects and a clean, cool, fresh feeling in the mouth.

A large number of compounds providing cooling sensations have been described. The most well-known natural occurring compound is menthol, in particular L-menthol. Among the synthetic compounds providing cooling sensations, many are derivatives of or are structurally related to menthol, i. e. containing the cyclohexane moiety, and derivatized with functional groups including carboxamide, ketal, ester, ether and alcohols.

Applicant surprisingly found a new class of chemical compounds which differ significantly in structural terms from the TRPM8 modulators known hitherto. It was surprisingly found that this class of chemical compounds as herein further described can provide long lasting cooling on the human skin and/or mucosa at very low concentrations.

### SUMMARY

There is provided in a first aspect an oxalate of a compound of formula (I) (in particular for use in providing cooling senstions)

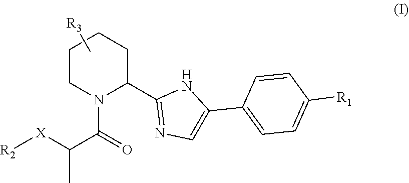

wherein

R₁ is selected from methyl, —CH₂OH, and —C(O)OH,

R₂ is selected from methyl, ethyl, and —CH₂—OH,

R₃ is selected from hydrogen and —OH, and

X is selected from CH₂, S, >S═O, and >SO₂.

In accordance with a second aspect there is provided a method of modulating (in-vitro and in-vivo modulation) of transient receptor potential melastatin member 8 (TRPM8) comprising bringing the receptor into contact with an oxalate of a compound of formula (I), or a solvate.

There is provided in a third aspect a method of inducing a cooling sensation in a human or animal comprising contacting the human or animal with an oxalate of a compound of formula (I), or solvate thereof.

There is provided in a fourth aspect consumer products, in particular consumer products which get into contact with the human skin and/or mucosa comprising an oxalate of a compound of formula (I), or solvate thereof.

There is provided in a fifth aspect a composition comprising a cool sensation wherein the composition comprises an oxalate of a compound of formula (I), or solvate thereof, and a further cooling compound.

There is provided in a sixth aspect pharmaceutical composition comprising an oxalate of a compound for formula (I), or solvate thereof.

There is provided in a seventh aspect a method of purification by crystallization of a compound of formula (I), comprising the step
a) admixing a compound of formula (I) with oxalic acid in the presence of an alcoholic solvent (e.g., 1-butanol, 2-butanol, 1-propanol, 2-propanol, 1-pentanol, 3-methyl-1-butanol, iso-butanol)
b) followed by the addition of an anti-solvent (e.g. n-heptane), and
c) separation of the oxalate of the compound of formula (I).

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based, at least in part, on the surprising finding of a new class of chemical compounds which differ significantly in structural terms from the TRPM8 modulators known hitherto, that are capable to activate the TRPM8 ion channel, which brings about a Ca²⁺influx into the cold-sensitive neurons. The electrical signal produced as a result is ultimately perceived as sensation of coldness. Applicant surprisingly found that this class of chemical compounds as herein further described can provide long lasting cooling on the human skin and/or mucosa at very low concentrations.

Thus, there is provided in a first aspect an oxalate of a compound of formula (I) (in particular for use in providing cooling sensations)

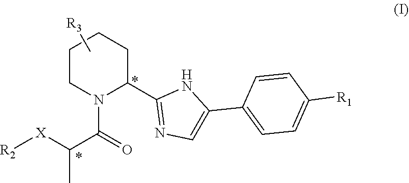

wherein

R₁ is selected from methyl, —CH₂OH, and —C(O)OH,

R₂ is selected from methyl, ethyl, and —CH₂—OH,

R₃ is selected from hydrogen and —OH, and

X is selected from CH₂, S, >S═O, and >SO₂.

Non-limiting examples are oxalates of a compound of formula (I) wherein X is S, R₁ is selected from methyl, —CH₂OH, and —C(O)OH, and R₂ is selected from methyl and ethyl.

Further non-limiting examples are oxalates of a compound of formula (I) wherein R₂ is methyl, R₁ is selected from methyl, —CH₂OH, and —C(O)OH, and X is selected from S, >S═O, and >SO₂.

Further non-limiting examples are oxalates of a compound of formula (I) wherein R₁ is methyl, R₂ is selected from methyl and ethyl, and X is selected from S, >S═O, and >SO₂.

Further non-limiting examples are oxalates of a compound of formula (I) wherein R₃ is hydrogen.

Further non-limiting examples are oxalates of a compound of formula (I) wherein R₁ is methyl, R₂ is selected from methyl and ethyl, R₃ is hydrogen, and X is selected from S, >S═O, and >SO₂.

As one specific example of an oxalate of a compound of formula (I) one may cite 2-(1-(2-(methylthio)propanoyl) piperidin-2-yl)-5-(p-tolyl)-1H-imidazolium carboxyformate (oxalate of 2-(methylthio)-1-(2-(5-(p-tolyl)-1H-imidazol-2-yl) piperidin-1-yl)propan-1-one).

Further non-limiting examples are the oxalate of a compound of formula (I) selected from the group consisting of
1-(2-(5-(4-(hydroxymethyl)phenyl)-1H-imidazol-2-yl)piperidin-1-yl)-2-(methylthio)propan-1-one;
4-(2-(1-(2-(methylthio)propanoyl)piperidin-2-yl)-1H-imidazol-5-yl)benzoic acid;
2-(methylsulfinyl)-1-(2-(5-(p-tolyl)-1H-imidazol-2-yl)piperidin-1-yl)propan-1-one;
1-(2-(5-(4-(hydroxymethyl)phenyl)-1H-imidazol-2-yl)piperidin-1-yl)-2-(methylsulfinyl)propan-1-one;
4-(2-(1-(2-(methylsulfinyl)propanoyl)piperidin-2-yl)-1H-imidazol-5-yl)benzoic acid;
2-(methylsulfonyl)-1-(2-(5-(p-tolyl)-1H-imidazol-2-yl)piperidin-1-yl)propan-1-one;
1-(2-(5-(4-(hydroxymethyl)phenyl)-1H-imidazol-2-yl)piperidin-1-yl)-2-(methylsulfonyl)propan-1-one; and
4-(2-(1-(2-(methylsulfonyl)propanoyl)piperidin-2-yl)-1H-imidazol-5-yl)benzoic acid.

By “oxalate” we mean the salt of oxalic acid wherein the molar ratio of oxalic acid to the compound of formula (I) is either 1:1 or 2:1.

The compounds as defined by formula (I) comprise several chiral centers (two of which are indicated by * in formula (I)) and as such may exist as a mixture of stereoisomers, or they may be resolved as isomerically pure forms. Resolving stereoisomers adds to the complexity of manufacture and purification of these compounds and so it is preferred to use the compounds as mixtures of their stereoisomers simply for economic reasons. However, if it is desired to prepare individual stereoisomers, this may be achieved according to methods known in the art, e.g. preparative HPLC and GC, crystallization or stereoselective synthesis. The compounds as defined by formula (I) may also exist in various tautomeric forms, including the 1H-Imidazole-3H-Imidazole form. Accordingly, the chemical structures depicted herein encompass all possible sterioisomers and tautomeric forms of the illustrated compounds.

The oxalates of the compounds as defined by formula (I) are “TRPM8 agonist”, which means that they have an agonistic effect on the cellular Ca²⁺ ion permeability of the TRPM8 channels. Accordingly, by “TRPM8 agonist” is meant any compound, which when brought into contact with the TRPM8 receptor, produces an increase in fluorescence over background, using the FLIPR method as described, e.g., by Klein et al., (Chem. Senses 36: 649-658, 2011), which is also described in more details in the experimental part.

Accordingly there is provided in a second aspect a method of modulating (in-vitro and in-vivo modulation) of transient receptor potential melastatin member 8 (TRPM8) comprising bringing the receptor into contact with an oxalate of a compound of formula (I).

In certain embodiments of the second aspect of the invention the modulating method is an in-vitro method.

There is provided in a third aspect a non-medical method of inducing a cooling sensation in a human or animal comprising contacting the human or animal with an oxalate of a compound of formula (I).

In certain embodiments, the method is a method of achieving a cooling effect on the skin or mucosa comprising contacting the skin or mucosa with a product comprising an oxalate of a compound of formula (I).

The oxalate of a compound of formula (I) may be applied directly or as a solution or suspension, comprising an effective amount of an oxalate of a compound of formula (I). An amount to be effective depends, inter alia, upon the target TRPM8 area of the body but also on the cooling potency of compound or mixture of compounds.

There is provided in a fourth aspect consumer products, in particular consumer products which get into contact with the human skin and/or mucosa comprising an oxalate of a compound of formula (I).

Consumer products which get in contact with the mucosa include, but are not limited to food products, beverages, chewing gum, tobacco and tobacco replacement products, dental care products, personal care products, including lip care products, sexual health and intimate care products.

In certain embodiments dental care products are oral care products, tooth care products, cleaners for dental prostheses, adhesives for dental prostheses, and the like.

In certain embodiments food products are iced consumable products such as ice cream, sorbet; confectioneries such as (hard) candies and chocolates; food products containing mint or mint flavour, sauces, dairy products such as milk-based drinks and yoghurts; and snacks.

In certain embodiments tobacco replacement products are liquids or solids which are suitable to be consumed by electrical means, e.g., liquids to vape.

In certain embodiments personal care products getting in contact with the mucosa are lip balms, nose sprays and eye drops.

Consumer products which get in contact with the human skin include, but are not limited to cosmetic products. In certain embodiments cosmetic products are skincare products, especially bath products, skin washing and cleansing products, skincare products, eye makeup, nail care products, foot care products, and the like. In certain embodiments cosmetic products are products with specific effects, especially sunscreens, insect repellent products, tanning products, de-pigmenting products, deodorants, antiperspirants, hair removers, and shaving products. In a certain embodiments cosmetic products are hair care products, especially hair shampoos, hair care products, hair setting products, hair-shaping products, and hair coloring products as well as scalp-care products such as scalp-cooling shampoos and creams.

In certain embodiments, the consumer product is selected from air care products, such as an air freshener or a “ready to use” powdered air freshener which can be used in the home space (rooms, refrigerators, cupboards, shoes or car) and/or in a public space (halls, hotels, malls, etc . . . ).

The consumer products can be in any physical form, such as a solid, semi-solid, plaster, solution, suspension, lotion, cream, foam, gel, paste, or a combination thereof. The physical form of the consumer product suitable manly depends on the specific actions, such as cleaning, softening, caring, cooling, and the like, such a consumer product should fulfill.

In a certain embodiment consumer products getting in contact with the human skin are fabric care products (such as fabric detergents, fabric conditioner (including tumble dryer sheets), and scent boosters (liquid or solid)) which in a first step are applied to a fabric, e.g., when washing the fabric, said treaded fabrics then getting in contact with the human skin.

The level of use for oxalates of a compound of formula (I) depend, inter alia, upon the target TRPM8 area of the body but also on the cooling potency of the oxalate. For examples in an oral application of an oxalate of a compound of formula (I), such as dentifrice, floss, chewing gum, or white strip, the levels of use may be from about 0.00001% (0.01 ppm) to about 0.1% (1000 ppm); from about 0.00005% (0.5 ppm) to about 0.1% (1000 ppm); from about 0.0001% (1 ppm) to about 0.05% (500 ppm); or from about 0.001% (10 ppm) to about 0.01% (100 ppm) by weight of the composition. When a compound of the present invention is used in a mouthwash, the level of use may be from about 0.000001% (10 ppb) to about 0.01% (100 ppm) or from about 0.0001% (1 ppm) to about 0.001% (10 ppm) by weight of the composition. When an oxalate of a compound of formula (I) is delivered topically, for example in shampoos and lotions the levels may be from about 0.001% (10 ppm) to about 0.5% (5000 ppm) by weight of the composition or from about 0.01% (100 ppm) to about 0.4% (4000 ppm) by weight of the composition.

The cooling potency (strength) of a compound is defined by its EC₅₀ value. EC₅₀ (half maximal effective concentration) refers to the concentration of a compound which induces a response halfway between the baseline and maximum after a specified exposure time. It is commonly used as a measure of potency. EC₅₀ is a measure of concentration, expressed in μM (μmolar) unites, where 1 μM is equivalent to 1 μmol/L.

Compounds with an EC₅₀ of 10 μM or less are perceived by the human as cooling. The lower the EC₅₀ value the higher the cooling potency. For example, compounds having an EC₅₀ value of about 0.1 μM are perceived as strong cooling compounds.

Cooling properties of a compound however are not only defined by its strength (potency; EC50) but also its longevity, which refers to the period of time (in minutes) over which a cooling effect is perceived. The longevity can range from a few seconds after rinsing to several hours or even days. In the context of oral care products, a preferred “long-lasting” effect ranges typically between 20 minutes after rinsing to 3 hours.

The oxalates of the compounds of formula (I) are very potent at relative low concentrations. Thus a stock solution might be prepared which is further diluted, before admixing it to a consumer product. Beside water, particular suitable solvents are triacetin and propylene glycol. One may also mention acetone, benzyl alcohol, dihydrolevoglucosenone, methyl-tetrahydrofuran, pentylene glycol, ethylene glycol, ethyl lactate, methyl lactate, propyl lactate, dimethylsulfoxide, ethanol, ethyl acetate, ethylene glycol, diethylene glycol, propylene glycol, and triacetin which are suitable solvents for oxalates of the compounds of formula (I). But other solvent systems comprising surfactants may also be used.

To modify the cooling effect of an oxalate of a compound of formula (I), the oxalate may be combined with a compound selected from calcium ions and salts, magnesium ions and salts, arginine, or any chelating agent which is able to bind calcium or magnesium.

These compounds are known to be able to modulate the concentration of such ions in the extracellular space and therefore influence the response of the TRPM8 ion-channel, leading to a change in the perceived cooling effect.

According to Kizilbash et al. (WO2019/121193 A1) both, the cooling intensity and the flavour intensity may be enhanced when combined with agents which possess the property to potentiating said effects. Thus the oxalates of the present invention may be combined in one particular embodiment with potentiating agents disclosed in WO2019/121193 which is incorporated by reference, in particular with regard to the potentiating agents.

As a further enhancement agent one may cite N-lactoyl ethanolamine (2-hydroxy-N-(2-hydroxyethyl) propanamide; CAS 5422-34-4) which is known as an enhancer for cooling agents, for example, from PCT International publication WO 2008/107137 which is incorporated by reference, in particular with regard to the cooling enhancing substances as defined by formula (I).

The oxalates of the present invention might be used in combination with other cooling compounds known in the art.

Thus there is provided in a fifth aspect a composition comprising a cool sensation wherein the composition comprises at least one oxalate of a compound of formula (I) and a further cooling compound.

In one particular embodiment the oxalates of the present invention may be combined with menthol (e.g., in form of peppermint oil, and/or spearmint oil), menthone, p-menthanecarboxamides, N-2,3-trimethyl-2-isopropyl-butanamide (WS-23), menthyl lactate (Frescolat® ML), menthone glycerol acetal (Frescolat® MGA), 3-(1-menthoxy)-propane-1,2-diol (TK-10), p-menthane-3,8-diol (known as Coolact 38D), isopulegol (known as Coolact P), monomenthyl succinate (Physcool®), monomenthyl glutarate, o-menthylglycerol, menthyl N,N-dimethylsuccinamate, 2-(sec-butyl)cyclohexan-1-one (Freskomenthe), N-(pyrazol-3-yl)-N-(thiophen-2-ylmethyl)-2-(p-tolyloxy) acetamide, 2-(4-ethylphenoxy)-N-(pyrazol-3-yl)-N-(thiophen-2-ylmethyl) acetamide, 3-(benzo[d][1,3]dioxol-5-yl)-N,N-diphenylacrylamide, 4-(2-(4-allyl-2,6-dimethoxyphenoxy)-1-ethoxypropyl)-2-methoxyphenol, 4-(2-(4-allyl-2,6-dimethoxyphenoxy)-1-((2-isopropyl-5-methylcyclohexyl)oxy)propyl)-2-methoxyphenol (including 4-(2-(4-allyl-2,6-dimethoxyphenoxy)-1-(((1S,2R,5S)-2-isopropyl-5-methylcyclohexyl)oxy)propyl)-2-methoxyphenol) and 4-(2-(4-allyl-2,6-dimethoxyphenoxy)-1-(((1R,2S,5R)-2-isopropyl-5-methylcyclohexyl)oxy)propyl)-2-methoxyphenol), N-(2-Hydroxy-2-phenylethyl)-2-isopropyl-5,5-dimethylcyclohexane-1-carboxamide, N-(4-(Cyanomethyl)phenyl)-2-isopropyl-5,5- dimethylcyclohexanecarboxamide and N-(3-Hydroxy-4-methoxyphenyl)-2-isopropyl-5,5-dimethylcyclohexanecarboxamide.

Examples of p-methanecarboxamides include compounds such as N-ethyl-p-menthan-3-carboxamide (known commercially as WS-3), N-ethoxycarbonylmethyl-p-menthan-3-carboxamide (WS-5), N-(4-methoxyphenyl)-p-menthan-3-carboxamide (WS-12) and N-tert-butyl-p-menthan-3-carboxamide (WS-14), N-(4-(cyanomethyl)phenyl)-2-isopropyl-5-methylcyclohexane-1-carboxamide (known commercially as Evercool 180), 2-isopropyl-5-methyl-N-(2-(pyridin-2-yl)ethyl)cyclohexane-1-carboxamide (known commercially as Evercool 190), and (1R,2S,5R)-N-((S)-2-((R)-2-aminopropanamido)-2-phenylethyl)-2-isopropyl-5-methylcyclohexane-1-carboxamide.

In order to achieve more than just a cooling effect, the oxalate of the present invention may be combined with other actives, such as, flavours, fragrances, and sweetening agents.

Examples of flavour ingredients include natural flavors, artificial flavors, spices, seasonings, and the like. Exemplary flavor ingredients include synthetic flavor oils and flavoring aromatics and/or oils, oleoresins, essences, and distillates, and a combination comprising at least one of the foregoing.

Flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavoring agents include artificial, natural and synthetic fruit flavors such as vanilla, and citrus oils including lemon, orange, lime, grapefruit, yuzu, sudachi, and fruit essences including apple, pear, peach, grape, raspberry, blackberry, gooseberry, blueberry, strawberry, cherry, plum, prune, raisin, cola, guarana, neroli, pineapple, apricot, banana, melon, apricot, cherry, tropical fruit, mango, mangosteen, pomegranate, papaya, and so forth.

Additional exemplary flavors imparted by a flavoring composition include a milk flavor, a butter flavor, a cheese flavor, a cream flavor, and a yogurt flavor; a vanilla flavor; tea or coffee flavors, such as a green tea flavor, an oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a chamomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; a nut flavor such as an almond flavor, a hazelnut flavor, a macadamia nut flavor, a peanut flavor, a pecan flavor, a pistachio flavor, and a walnut flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor.

Generally any flavoring or food additive (including food colors) such as those described in “Essential guide to food additives”, Third edition2008, page 101-321 (ISBN: 978-1-905224-50-0) by Leatherhead Food International Ltd., can be used. The publication is incorporated herein by reference.

In one particular embodiment the oxalates of the present invention may be combined with anethole, menthol laevo, carvone laevo, ethyl maltol, vanillin, eucalyptol, eugenol, menthol racemic, cis-3-hexenol, linalol, mint oil (e.g. peppermint arvensis oil (e.g. including Japanese peperming oil, and American peppermint oil), peppermint piperita oil, spearmint native oil, spearmint scotch oil), corylone, ethyl butyrate, cis-3-hexenyl acetate, citral, eucalyptus oil, ethyl-vanillin, methyl salicylate, 2′-hydroxypropiophenone, ethyl acetate, methyl dihydro jasmonate, geraniol, lemon oil, iso amyl acetate, thymol, ionone beta, linalyl acetate, decanal, cis jasmone, ethyl hexanoate, melonal (2,6-dimethylhept-5-enal), citronellol, ethyl aceto acetate, nutmeg oil and clove oil, or mixtures thereof.

Examples of sweetening agents include, but are not limited to, sucrose, fructose, glucose, high fructose corn syrup, corn syrup, xylose, arabinose, rhamnose, erythritol, xylitol, mannitol, sorbitol, inositol, acesulfame potassium, aspartame, neotame, sucralose, and saccharine, and mixtures thereof; trilobatin, hesperetin dihydrochalcone glucoside, naringin dihydrochalcone, mogroside V, Luo Han Guo extract, rubusoside, rubus extract, glycyphyllin, isomogroside V, mogroside IV, siamenoside I, neomogroside, mukurozioside IIb, (+)-hernandulcin, 4 β-hydroxyhernandulcin, baiyunoside, phlomisoside I, bryodulcoside, bryoside bryonoside, abrusosides A-E, cyclocarioside A, cyclocaryoside I, albiziasaponins A-E, glycyrrhizin, araboglycyrrhizin, periandrins I-V, pterocaryosides A and B, osladin, polypodosides A and B, telosmoside A8-18, phyllodulcin, huangqioside E neoastilbin, monatin, 3-acetoxy-5,7-dihydroxy-4′-methoxyflavanone, 2R,3R-(+)-3-Acetoxy-5,7,4′-trihydroxyflavanone, (2R,3R)-dihydroquercetin 3-O-acetate, dihydroquercetin 3-O-acetate 4′-methyl ether, brazzein, curculin, mabinlin, monellin, neoculin, pentadin, thaumatin, and combinations thereof. Some of the compounds listed above are known sweetness enhancers as well as sweeteners. When used as sweetness enhancers they are normally used below their sweetness detection thresholds.

In certain embodiments, the oxalates of the present invention may be combined with additional ingredients collectively refereed to orally acceptable carrier materials.

In some aspects, the orally acceptable carrier may comprise one or more compatible solid or liquid excipients or diluents which are suitable for topical oral administration. By “compatible,” as used herein, is meant that the components of the composition are capable of being commingled without interaction in a manner which would substantially reduce stability and/or efficacy. The carriers can include the usual and conventional components of dentifrices, non-abrasive gels, subgingival gels, mouthwashes or rinses, mouth sprays, chewing gums, lozenges and breath mints. The choice of a carrier to be used is basically determined by the way the composition is to be introduced into the oral cavity. Carrier materials for toothpaste, tooth gel or the like include abrasive materials, sudsing agents, binders, humectants, flavoring and sweetening agents, etc. as disclosed in e.g., U.S. Pat. No. 3,988,433, to Benedict. Carrier materials for biphasic dentifrice formulations are disclosed in U.S. Pat. Nos. 5,213,790; 5,145,666 and 5,281,410 all to Lukacovic et al., and in U.S. Pat. Nos. 4,849,213 and 4,528,180 to Schaeffer. Mouthwash, rinse or mouth spray carrier materials typically include water, flavoring and sweetening agents, etc., as disclosed in, e.g., U.S. Pat. No. 3,988,433 to Benedict. Lozenge carrier materials typically include a candy base; chewing gum carrier materials include a gum base, flavoring and sweetening agents, as in, e.g., U.S. Pat. No. 4,083,955, to Grabenstetter et al. Sachet carrier materials typically include a sachet bag, flavoring and sweetening agents. For subgingival gels used for delivery of actives into the periodontal pockets or around the periodontal pockets, a “subgingival gel carrier” is chosen as disclosed in, e.g. U.S. Pat. Nos. 5, 198,220 and 5,242,910 both to Damani. Carriers suitable for the preparation of compositions of the present disclosure are well known in the art. Their selection will depend on secondary considerations like taste, cost, and shelf stability, and the like.

Further suitable types of orally acceptable carrier materials or excipients are listed in WO2010/059289, in particular on page 17-31, which is incorporated by reference.

Scientific literature points out that the activation of TRPM8 channels may be useful for the treatment of most TRPM8-mediated pathologies (J. Med. Chem. 2016, 59 (22), 10006-10029). Thus one may assume that the oxalates of the present invention might also be suitable for treating prostate carcinomas, bladder weakness, inflammation, or pain comprising contacting a patient with an oxalate of a compound of formula (I) as defined herein. One may also assume that the oxalates are suitable for alleviating the symptoms of coughs and colds, irritations, sore throat or hoarseness, as well as the treatment of laryngopharyngeal dysphagia (Int. J. Mol. Sci. 2018, 19, 4113).

Thus there is provided in a sixth aspect pharmaceutical composition comprising an oxalate of a compound of formula (I) as hereinabove defined.

Depending upon the particular treatment regimen contemplated, pharmaceutical compositions comprising an oxalate of a compound of formula (I) may be administered parenterally, topically, orally, or locally. The pharmaceutical compositions may be a liquid, suspensions or a solid formulation.

In certain embodiments, the pharmaceutical composition is nasal spray, topical cream, skin sprays, throat spray, or eye drops.

The oxalates of a compound of formula (I) are not described in the literature and thus are novel in their own right.

Thus, there is provided in a further aspect of the invention an oxalate of a compound of formula (I)

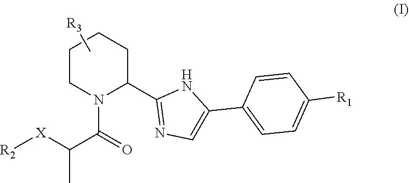

wherein

R₁ is selected from methyl, —CH₂OH, and —C(O)OH,

R₂ is selected from methyl, ethyl, and —CH₂—OH,

R₃ is selected from hydrogen and —OH, and

X is selected from CH₂, S, >S═O, and >SO₂.

The compounds of formula (I) can be generally prepared as described in the international patent application PCT/EP2020/079009 (WO 2021074281) which is incorporated by reference.

Whereas certain compounds of formula (I) are amorphous and thus difficult to purify and thus, for example, difficult to dose, the oxalates of the compounds of formula (I) are accessible in very pure form due to their crystalline form at room temperature.

Thus, there is provided in a seventh aspect of the invention a method of purification by crystallization of a compound of formula (I), comprising the step
a) admixing a compound of formula (I) with oxalic acid in the presents of an alcoholic solvent,
b) followed by the addition of an anti-solvent, and
c) separation of the oxalate of the compound of formula (I).

In one particular embodiment the compounds of formula (I) can be admixed with oxalic acid without prior purification. Preferably at least 1 mol equivalent (e.g. 1.33 mol equivalents) of oxalic acid is added to convert the entire amount of the compound of formula (I) present in the (crude) mixture.

Suitable alcoholic solvents may be selected from 1-butanol, 2-butanol, 1-propanol, 2-propanol, 1-pentanol, 3-methyl-1-butanol, isobutanol. In one specific embodiment the alcoholic solvent is butanol (e.g. 1-butanol).

By “anti-solvent” we mean a solvent in which the oxalate of the compound of formula (I) is less soluble. As specific examples one may mention aliphatic hydrocarbons and aromatic solvents, such as, n-heptane, hexane, cyclohexane, pentane, toluene, and xylene, and mixtures thereof. In one specific embodiment the anti-solvent is n-heptane.

The anti-solvent is added in the second step to mediate the crystallization. The amount of anti-solvent (e.g. n-heptane) added in step b) should preferably not exceed the amount of the alcoholic solvent (e.g. 1-butanol) added in step a). In one particular embodiment alcoholic solvent (e.g. 1-butanol) should be added at about 4-5 times of the volume of the crude mixture comprising the compound of formula (I).

To initiate the crystallization one may add a minor amount of an oxalate of the compound of formula (I) after the compound of formula (I) and oxalic acid is completely dissolved in 1-butanol.

The separation step c) can be performed by means of, e.g. filtration of the oxalate or evaporation of the solvent. The filtration of the oxalated is preferred to obtaining purer crystals.

In one particular embodiment there is provided a method of purification by crystallization of a compound of formula (I), comprising the step
a) admixing a compound of formula (I) with oxalic acid in the presents of 1-butanol,
b) followed by the addition of n-heptane, and
c) separation of the oxalate of the compound of formula (I).

In this particular embodiment the crystallization step b) is preferably initiated at about 50° C. followed by cooling to about 0° C. to obtain an oxalate having almost the same stereoisomer composition as the compound of formula (I) added in step a). A higher initial temperature, such as about 60-65° C., might result in the (partial) loss of one diastereomer.

The invention is now further described with reference to the following non-limiting examples. These examples are for the purpose of illustration only and it is understood that variations and modifications can be made by one skilled in the art.

### EXAMPLES

### Example 1: Oxalate of 2-(methylthio)-1-(2-(5-(p-tolyl)-1H-imidazol-2-yl)piperidin-1-yl)propan-1-one

### Example 1a: 1-(2-(methylthio)propanoyl)piperidine-2-carboxylic Acid

A 2000 ml Ika reactor, equipped with an automated pH adjustment, a thermostat and a dropping funnel was charged with NaOH 2M (683 ml, 0.6 mol. eq.). Pipecolic acid (300 g, 2.276 mol) was added in portions follwed by additional NaOH 2M (240 ml). The mixture was cooled to 5° C. and 2-(methylthio)propanoyl chloride (325 g, 2.276 mol) was added over 2 hours whilst maintaining a pH of 13. The mixture was stirred at 5° C. for one hour, HCl 2M (2500 ml) was added and the aqueous solution was extracted with MtBE (2×1500 ml). The combined organic layers were washed with water (3×1500 ml) and brine (500 ml). The organic phase was dried over MgSO₄ and concentrated under reduced pressure. Crude 1-(2-(methylthio)propanoyl)piperidine-2-carboxylic acid (480 g) was obtained as yellow viscous oil. The combined crude products from two batches (951 g) were recrystallized from MtBE to give 1-(2-(methylthio)propanoyl)piperidine-2-carboxylic acid (570 g, 2.46 mol, 54% yield) as a white solid.

### Example 1b: 2-oxo-2-(p-tolyl)ethyl 1-(2-(methylthio)propanoyl)piperidine-2-carboxylate

A 4500 ml sulfonating flask, equipped with an overhead stirrer and heated with an oil bath, was charged with tributylamine (961 g, 5.133 mol) and tretrabutylammonium bromide (76 g, 0.234 mol). The mixture was heated to 70° C. internal temperature and 1-(2-(methylthio)propanoyl)piperidine-2-carboxylic acid (1197 g, 5.123 mol) was added in portions over 30 minutes. Then 2-chloro-1-(p-tolyl)ethan-1-one (815 g, 4.783 mol) was added in portions over 20 minutes and the reaction mixture was stirred at 90° C. for 30 minutes. The reaction mixture was cooled to 55° C., MtBE (1000 ml) was added and the reaction was quenched with water (1400 ml). The organic phase was separated and washed with HCl 2M (1200 ml) and water (1500 ml). After addition of NaOH 2M, the organic phase was separated and washed with water (3×1500 ml) and brine (1000 ml). The organic phase was dried over MgSO₄ and the product left to crystallized for 16 hours. The solids were isolated by filtration, washed with cold MtBE and pentane and dried under reduced pressure to give 2-oxo-2-(p-tolyl)ethyl 1-(2-(methylthio)propanoyl)piperidine-2-carboxylate (1409 g, 81% yield) as white crystals.

Example 1c: 2-(methylthio)-1-(2-(5-(p-tolyl)imidazol-2-yl)piperidin-1-yl)propan-1-one: 2-oxo-2-(p-tolyl)ethyl 1-(2- (methylthio)propanoyl)piperidine-2-carboxylate (550 g, 1.513 mol) was placed in a 2500 ml sulfonating flask equipped with a Dean-Stark water separator, mechanical stirrer and heated by an oil bath. Toluene (700 ml) was added and the mixture was heated to reflux (temperature of the oil bath 140° C.). Ammonium acetate (116 g, 1.513 mol) was added in one portion. Strong gas evolution was observed and water produced by the reaction started to separate. After 45 minutes another portion of ammonium acetate (145 g, 1.891 mol) was added followed by another portion (145 g, 1.891 mol) 45 minutes later. After adding a total of 3.5 equivalents of ammonium acetate the mixture was stirred under reflux for 1 hour, cooled to 60° C. and the pH set to 7 by adding NaOH 2M (ca. 800 ml). The organic phase was separated and the aqueous phase extracted with MtBE (500 ml). The combined organic phases were then concentrated to give 565 g of a dark-orange, viscous crude quality of 2-(methylthio)-1-(2-(5-(p-tolyl)imidazol-2-yl)piperidin-1-yl)propan-1-one. Purification of a small sample by silica gel chromatography (gradient of EtOAc in Heptane) furnished 2-(methylthio)-1-(2-(5-(p-tolyl)imidazol-2-yl)piperidin-1-yl)propan-1-one as a white solid. 20)

MS (EI, 70 eV): 343 (2, [M]+·), 241 (17), 240 (100), 213 (13), 185 (18), 184 (9), 75 (55), 56 (11), 55 (9), 47 (10), 41 (11). ¹H NMR (400 MHZ, DMSO-d₆, mixture of stereoisomers and tautomers) δ 12.07, 11.99, 11.95, 11.76 (brs, 1H), 7.72-7.60 (m, 2H), 7.59-7.42 (m, 1H), 7.26-7.08 (m, 2H), 5.75-5.39 (m, 1H), 4.49-3.00 (m, 3H), 2.71-2.15 (m, 1H), 2.30 (s, 3H), 2.07-1.96 (m, 3H), 1.94-1.48 (m, 5H), 1.43-1.34 (m, 3H) ppm. ¹³C NMR (101 MHZ, DMSO-d₆, mixture of stereoisomers and tautomers) δ 170.2 (q), 170.2 (q), 170.0 (q), 147.0 (q), 146.8 (q), 146.7 (q), 140.4 (q), 140.3 (q), 139.9 (q), 135.3 (q), 135.2 (q), 135.1 (q), 132.7 (q), 132.6 (q), 132.6 (q), 129.7 (t), 129.3 (t), 124.6 (t), 113.0 (t), 112.6 (t), 112.5 (t), 51.5 (t), 47.3 (t), 47.0 (t), 43.1 (d), 43.0 (d), 38.8 (d), 38.0 (t), 37.6 (t), 37.3 (t), 28.8 (d), 28.6 (d), 28.1 (d), 27.9 (d), 26.1 (d), 25.7 (d), 25.4 (d), 21.2 (s), 20.3 (d), 20.1 (d), 18.3 (s), 18.0 (s), 17.8 (s), 11.9 (s), 11.7 (s), 11.6 (s) ppm.

### Example 1d: Oxalate of 2-(methylthio)-1-(2-(5-(p-tolyl)imidazol-2-yl)piperidin-1-yl)propan-1-one

Crude 2-(methylthio)-1-(2-(5-(p-tolyl)imidazol-2-yl)piperidin-1-yl)propan-1-one prepared as described in Example 1a (48.7 g) was suspended in 1-butanol (165 ml, 4.5 vols). The mixture was warmed to 65° C. and oxalic acid (9.56 g, 1.33 mol equiv.) was charged portion wise over 9 minutes, maintaining a temperature of 60-65° C. Upon complete addition, a vessel rinse of 1-butanol (17.5 ml, 0.5 vols) was applied and the dark orange/brown solution cooled to 50° C. 2-(1-(2-(Methylthio)propanoyl)piperidin-2-yl)-5-(p-tolyl)-1H-imidazolium carboxyformate seed (365 mg, 1% wt.) was charged and observed to hold in solution. The mixture was held at 50° C. for 1 hour, before heptane (184 ml, 5 vols) was charged drop wise via syringe pump over 7 hours at 50° C. Upon complete addition, the mixture was held at 50° C. for 1 hour, cooled to 0° C. over 12.5 hours and then held at 0° C. for 1 hour before the solids were isolated by filtration. The vessel and filter cake were washed with heptane/1-butanol [3:1] (2×73 ml, 2×2 vols). The filter cake was air dried under reduced pressure for 60 minutes, the bulk solids were collected and dried under reduced pressure at 65° C. to yield the oxalate of 2-(methylthio)-1-(2-(5-(p-tolyl)imidazol-2-yl)piperidin-1-yl)propan-1-one (36.8 g, 848 mmol, 79.8% recovery) as off-white crystals.

Major conformer of diastereomer 1:

¹H NMR (600 MHZ, MeOD-d₄) δ 7.70 (br s, 1H), 7.63-7.58 (m, 2H), 7.32-7.28 (m, 2H), 6.14-6.08 (br m, 1H), 4.13-4.05 (br m, 1H), 3.93 (br q, J=6.8 Hz, 1H), 3.20-3.11 (br m, 1H), 2.57-2.51 (br m, 1H), 2.38 (s, 3H), 2.07 (s, 3H), 1.98-1.88 (br m, 1H), 1.86-1.77 (br m, 2H), 1.72-1.66 (br m, 1H), 1.57-1.48 (br m, 1H), 1.49 (d, J=6.4 Hz, 3H) ppm.

¹³C NMR (150 MHZ, MeOD-d₄) δ 173.6 (s), 166.3 (2s), 148.4 (s), 140.9 (s), 136.7 (s), 131.0 (2d), 127.2 (2d), 126.2 (s), 116.6 (d), 49.0 (d), 44.8 (t), 38.8 (d), 28.4 (t), 26.0 (t), 21.5 (q), 21.2 (t), 17.5 (q), 11.4 (q) ppm.

Major conformer of diastereomer 2 (shifts partially extracted from HSQC/HMBC):

¹H NMR (500 MHZ, MeOD-d₄) δ 7.72 (br s, 1H), 7.65-7.60 (m, 2H), 7.30-7.26 (br m, 2H), 5.87-5.83 (m, 1H), 4.66-4.59 (br m, 1H), 3.85 (br q, J=6.6 Hz, 1H), 2.60-2.53 (br m, 1H), 2.36 (s, 3H), 2.24-2.15 (br m, 1H), 2.07 (br s, 3H), 2.06-1.97 (br m, 1H), 1.85-1.76 (br m, 1H), 1.72-1.62 (br m, 1H), 1.59-1.49 (m, 1H), 1.50-1.46 (br m, 3H), 1.49-1.37 (br m, 1H) ppm.

¹³C NMR (150 MHz, MeOD-d₄) δ 171.9 (s), 166.3 (s), 148.0 (s), 140.7 (s), 137.0 (s), 131.0 (2d), 127.2 (2d), 126.2 (s), 116.8 (d), 53.1 (d), 40.8 (t), 38.8 (d), 28.4 (t), 26.3 (t), 21.6 (t), 21.5 (q), 17.7 (q), 11.3 (q) ppm.

### Example 2: Assay on TRPM8 Modulators

A HEK293 cell line stably expressing hTRPM8 was generated according to Klein et al., (Chem. Senses 36: 649-658, 2011) and receptor activation was monitored by calcium imaging in a Flexstation. For Ca-imaging assays of TRPM8 channel activation, cells were seeded on day 0 at a density of 12000 cells per well in Dulbecco's modified Eagle medium (DMEM) containing 9% foetal bovine serum in black, clear bottom 96-well plates that had been coated with 0.001% polyethyleneimine (molecular weight=60 000, Acros Organics). On day 2, agonists were evaluated via calcium imaging using Fluo-4. Briefly, growth medium was discarded, and the cells were incubated in the dark for 1 h at 37° C. in 50 μL loading buffer consisting of 2.7 μM Fluo-4 AM (Invitrogen) and 2.5 μM probenecid (Sigma-Aldrich) in DMEM (without serum). After incubation, the plates were washed five times with 100 μL of assay buffer (in mM: 130 NaCl, 5 KCl, 10 HEPES, 2 CaCl₂, and 10 glucose, pH7.4.) and further incubated in the dark at room temperature for 30 min. The cells were then washed five times with 100 μL assay buffer and then calcium influx to serial dilutions of 2-(1-(2-(methylthio)propanoyl)piperidin-2-yl)-5-(p-tolyl)-1H-imidazolium carboxyformate was measured in a Flexstation 3 (Molecular Devices). Receptor activation was initiated following addition of 20 μl of a 10-fold concentrated ligand stock solution, which is also prepared in assay buffer. Fluorescence was continuously monitored for 15 seconds prior to ligand addition and for 105 seconds after ligand addition, for a total of 120 seconds. Maximal receptor activation in relation to solvent control and relative to 31.6 μM menthol is determined. Data from serial dilutions were processed with a KNIME workflow to fit a sigmoidal dose-response curve and to extrapolate EC50 values.

The TRPM8 agonist 2-(1-(2-(methylthio)propanoyl)piperidin-2-yl)-5-(p-tolyl)-1H-imidazolium carboxyformate, which is the oxalate of 2-(methylthio)-1-(2-(5-(p-tolyl)-1H-imidazol-2-yl)piperidin-1-yl)propan-1-one″ exhibits and EC50 value below 0.01 μM.

### Example 3: Sensory Evaluation in Model Dentifrice

The oxalate of 2-(methylthio)-1-(2-(5-(p-tolyl)imidazol-2-yl)piperidin-1-yl)propan-1-one (prepared according to Example 1d) was dissolved at a concentration of 1 wt-% in propylene glycol. This solution was then dispersed in an appropriate quantity to obtain the desired final concentration of 10 ppm (parts per million) of the test compound into a model, unflavored dentifrice, the formula of which is given below (ingredients obtained from the suppliers respectively indicated in parenthesis).

| | wt % |
|---|---|
| Trisodium Phosphate dodecahydrate | 0.2 |
| Sorbitol | 50.0 |
| Precipitated Silica - Sorbosil ™ AC77 (Surfachem) | 8.0 |
| Precipitated Silica - Sorbosil ™ TC15 (Surfachem) | 9.0 |
| Sodium Carboxy Methyl Cellulose 9M31XF (Ashland) | 0.8 |
| Titanium Dioxide | 1.0 |
| Sodium Lauryl Sulphate | 1.5 |
| PEG 1500 (Kilo) | 5.0 |
| Demin Water | 24.5 |

A panel of trained flavourists evaluated the dentifrice containing the oxalate, by brushing their teeth with 1 g of the dentifrice using a toothbrush for 60 seconds, followed by spitting, without rinsing the mouth afterwards for the duration of the evaluation. The flavourists evaluated and recorded the cooling performance as well as other sensorial and organoleptic attributes at different time-points over a period of 90 minutes. The following tasting notes were recorded: Good coolant. Clean, with a pleasant, mild tingling effect. Active in all areas of the mouth. Fast acting (upfront cooling) with effect perceived already during brushing. Strong cooling peak with long-lasting effect up to one hour. No negative features or bitterness.”

1. A method of modulating of transient receptor potential channel melastatin member 8 (TRPM8) comprising bringing the receptor into contact with an oxalate of a compound of formula (I) 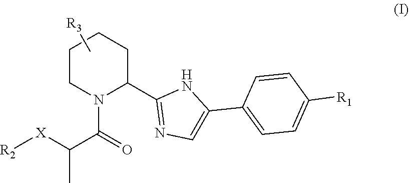
wherein
R₁ is selected from methyl, —CH₂OH, and —C(O)OH,
R₂ is selected from methyl, ethyl, and —CH₂—OH,
R₃ is selected from hydrogen and —OH, and
X is selected from CH₂, S,>S═O, and >SO₂.

2. A non-medical method of inducing a cooling sensation in a human or animal comprising contacting the human or animal with an oxalate of a compound of formula (I) as defined in claim 1.

3. A consumer product comprising an oxalate as defined in claim 1.

4. A pharmaceutical composition comprising an oxalate as defined in claim 1.

5. (canceled)

6. A medicament comprising the oxalate as defined in claim 1.

7. A composition for inducing a cooling sensation wherein the composition comprises an oxalate of a compound of formula (I) 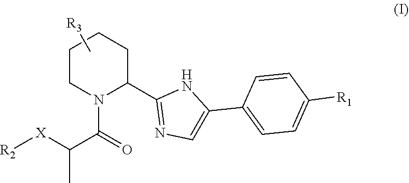
wherein
R₁ is selected from methyl, —CH₂OH, and —C(O)OH,
R₂ is selected from methyl, ethyl, and —CH₂—OH,
R₃ is selected from hydrogen and —OH, and
X is selected from CH₂, S, >S═O, and >SO₂.

8. The composition according to claim 7 comprising a further cooling compound.

9. The composition according to claim 8, wherein the further cooling compound is selected from the group consisting of menthol, menthone, p-menthanecarboxamides, N-2,3-trimethyl-2-isopropyl-butanamide, menthyl lactate, menthone glycerol acetal, 3-(1-menthoxy)-propane-1,2-diol, p-menthane-3,8-diol, isopulegol, monomenthyl succinate, monomenthyl glutarate, o-menthylglycerol, menthyl N,N-dimethylsuccinamate, 2-(sec-butyl)cyclohexan-1-one, N-(pyrazol-3-yl)-N-(thiophen-2-ylmethyl)-2-(p-tolyloxy)acetamide, 2-(4-ethylphenoxy)-N-(pyrazol-3-yl)-N-(thiophen-2-ylmethyl)acetamide, 3-(benzo[d][1,3]dioxol-5-yl)-N,N- diphenylacrylamide, 4-(2-(4-allyl-2,6-dimethoxyphenoxy)-1-ethoxypropyl)-2-methoxyphenol, 4-(2-(4-allyl-2,6-dimethoxyphenoxy)-1-((2-isopropyl-5-methylcyclohexyl)oxy)propyl)-2-methoxyphenol, N-(2-hydroxy-2-phenylethyl)-2-isopropyl-5,5-dimethylcyclohexane-1-carboxamide, N-(4-(cyanomethyl)phenyl)-2-isopropyl-5,5-dimethylcyclohexanecarboxamide, N-(3-hydroxy-4-methoxyphenyl)-2-isopropyl-5,5-dimethylcyclohexanecarboxamide, and mixtures thereof.

10. A method of purification by crystallization of a compound of formula (I), comprising the steps:
a) admixing a compound of formula (I) with oxalic acid in the presence of an alcoholic solvent,
b) followed by the addition of an anti-solvent, and
c) separation of the oxalate of the compound of formula (I)

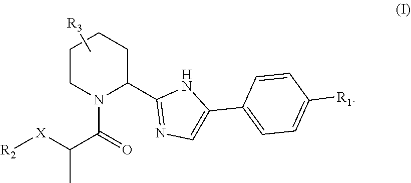

11. An oxalate of a compound of formula (I) 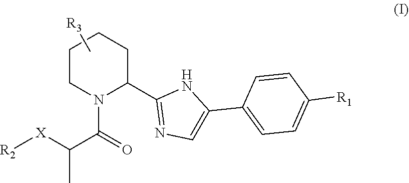
wherein
R₁ is selected from methyl, —CH₂OH, and —C(O)OH,
R₂ is selected from methyl, ethyl, and —CH₂—OH,
R₃ is selected from hydrogen and —OH, and
X is selected from CH₂, S, >S═O, and >SO₂.

12. The oxalate according to claim 11 selected from an oxalate of at least one of 2-(methylthio)-1-(2-(5-(p-tolyl)-1H-imidazol-2-yl)piperidin-1-yl)propan-1-one;
1-(2-(5-(4-(hydroxymethyl)phenyl)-1H-imidazol-2-yl)piperidin-1-yl)-2-(methylthio)propan-1-one; 4-(2-(1-(2-(methylthio)propanoyl)piperidin-2-yl)-1H-imidazol-5-yl)benzoic acid;
2-(methylsulfinyl)-1-(2-(5-(p-tolyl)-1H-imidazol-2-yl)piperidin-1-yl)propan-1-one;
1-(2-(5-(4-(hydroxymethyl)phenyl)-1H-imidazol-2-yl)piperidin-1-yl)-2-(methylsulfinyl)propan-1-one; 4-(2-(1-(2- (methylsulfinyl)propanoyl)piperidin-2-yl)-1H-imidazol-5-yl)benzoic acid; 2-(methylsulfonyl)-1-(2-(5-(p-tolyl)-1H-imidazol-2-yl)piperidin-1-yl)propan-1-one; 1-(2-(5-(4-(hydroxymethyl)phenyl)-1H-imidazol-2-yl)piperidin-1-yl)-2-(methylsulfonyl)propan-1-one; or 4-(2-(1-(2-(methylsulfonyl)propanoyl)piperidin-2-yl)-1H-imidazol-5-yl)benzoic acid.
